(19) United States
(12) Patent Application Publication
(10) Pub. No.: US 2025/0049847 A1
(43) Pub. Date: Feb. 13, 2025

(54) A METHOD FOR INDUCING IPSC DIFFERENTIATION TO OBTAIN CD34+CELLS AND NK CELLS AND APPLICATION THEREOF

(71) Applicant: ALLIFE MEDICINE (BEIJING) LIMITED, Beijing (CN)

(72) Inventors: Shixin GONG, Beijing (CN); Yuchun GU, Beijing (CN); Nan LI, Beijing (CN); Mingyue JIANG, Beijing (CN); Wenhua CAO, Beijing (CN); Qinqing PENG, Beijing (CN); Lida WU, Beijing (CN)

(73) Assignee: ALLIFE MEDICINE (BEIJING) LIMITED, Beijing (CN)

(21) Appl. No.: 18/565,841

(22) PCT Filed: Aug. 3, 2022
(86) PCT No.: PCT/CN2022/109959

§ 371 Date: Nov. 30, 2023

(30) Foreign Application Priority Data
Jun. 16, 2022 (CN) 202210677254.1

(51) Int. Cl. A61K 35/17; A61K 35/28; C12N 5/0783; C12N 5/0789

(52) U.S. Cl. CPC A61K 35/17; A61K 35/28; C12N 5/0646; C12N 5/0647; C12N 2500/02; C12N 2500/25; C12N 2500/32; C12N 2500/38; C12N 2501/115; C12N 2501/125; C12N 2501/145; C12N 2501/15; C12N 2501/155; C12N 2501/165; C12N 2501/2303; C12N 2501/2307; C12N 2501/2315; C12N 2501/26; C12N 2501/727; C12N 2506/45

(57) ABSTRACT

The present invention discloses a method and its application for inducing iPSC differentiation to obtain CD34+cells and NK cells. The method utilizes the three-dimensional structure of the embryoid bodies to provide a favorable differentiation microenvironment for iPSC differentiation, and can start producing a high proportion of CD34+cells on the 4th day under hypoxic induction culture conditions. Subsequently, the production of iPSC induced NK cell differentiation is significantly increased by adhering to the embryoid bodies wall or digesting and resuspension induction, And the induced differentiation of NK cells can exert a killing effect on tumor cells in a short period of time, with strong tumor killing ability, suitable for the production and clinical application of large-scale cell preparations.

### FIELD

The present invention belongs to the field of biopharmaceutical technology. Specifically, the present invention relates to a method for inducing iPSC differentiation to obtain CD34+cells and NK cells and its application.

### BACKGROUND

Hematopoietic stem cells (HSCs) are an extremely important type of stem cells in the adult body. Although they account for less than one in ten thousand blood cells in the human body, they possess strong self-renewal and differentiation abilities, and can long-term rebuild the entire blood and immune systems of the body, possessing the differentiation potential of blood and immune cells from various lineages. Hematopoietic stem cells are the earliest discovered type of stem cell in humans and the most studied type of stem cell to date. In recent years, with the development of hematopoietic stem cell transplantation treatment, the research on hematopoietic stem cells is increasingly in-depth. In clinical treatment, hematopoietic stem cell transplantation is widely used in hematological diseases and autoimmune diseases. In the treatment of other solid tumors, such as lymphoma, germ cell tumor, breast cancer, small cell lung cancer, it is mainly used in patients with conventional treatment failure or recurrence and refractory and with adverse prognostic factors. The main sources of hematopoietic stem cell transplantation are umbilical cord blood, bone marrow, and peripheral blood, but the proportion of hematopoietic stem cells is up to 1-5%, so it is necessary to obtain a sufficient number of hematopoietic stem cells through in vitro expansion.

CD34 molecule belongs to the cadherin family and is a highly glycosylated single transmembrane protein. It is selectively expressed on the surface of hematopoietic stem cells (HSCs), hematopoietic progenitor cells (HPCs), and endothelial cells (EC) in humans and other mammals, and gradually weakens to disappear with cell maturation. It is a typical surface marker for primary blood cells and bone marrow-derived progenitor cells (especially hematopoietic cells). CD34 protein is mainly expressed in hematopoietic stem cells and hematopoietic progenitor cells. At the same time, CD34 is also expressed on the surface of vascular endothelial cells and some mesenchymal stem cells. The expression intensity of CD34 is relatively high in the umbilical cord and bone marrow. Hematopoietic stem cell transplantation is mainly divided into two types: autologous and allogeneic hematopoietic stem cell transplantation. Although autologous transplantation has the advantages of no transplant rejection, no graft-versus-host disease, and other complications, the shortage of autologous hematopoietic stem cells stored in umbilical blood banks limits its clinical application in diseases. Although the long-term efficacy of allogeneic transplantation is better than that of autologous transplantation and the recurrence rate is low, the matching efficiency is extremely low and the source is limited, which limits its clinical application.

Therefore, there is an urgent need to seek safer, lower cost, and stable sources of hematopoietic stem cell resources in this field. Human pluripotent stem cells have the ability to differentiate into almost all types of somatic cells, including hematopoietic stem cells. Human pluripotent stem cells include human embryonic stem cells (ESC) and induced pluripotent stem cells (iPSC). Research has shown that human embryonic stem cells from mice, monkeys, and humans can be induced to differentiate into various blood cells in vitro. However, human embryonic stem cells originate from early embryonic development and face difficulties in obtaining materials, immune rejection, ethical and moral issues. Human induced pluripotent stem cells (iPSCs) can be reprogrammed from somatic cells such as human skin and blood in vitro, and have unlimited proliferation ability similar to human embryonic stem cells, as well as the ability to differentiate into almost all functional cells, including hematopoietic stem cells, in vitro. The characteristic of human induced pluripotent stem cells successfully bypasses the two most critical issues of immune rejection and ethics, providing the possibility of obtaining hematopoietic stem cells from in vitro sources for clinical transplantation applications.

At present, there are many methods for inducing human induced pluripotent stem cells to differentiate into CD34+cells, such as embryoid bodies (EB) differentiation methods, adherent induction differentiation methods, and the combination of different cytokines and compounds to induce the differentiation of CD34+ hematopoietic stem cells. However, the main drawbacks of the existing methods for inducing differentiation are long time to obtain CD34+cells and low yield. It can be seen that although there is some understanding in this field of the process of inducing pluripotent stem cells to differentiate into hematopoietic progenitor cells in vitro, existing differentiation methods still have some obvious shortcomings, including long induction differentiation time and low yield. Therefore, there is an urgent need to develop an efficient and rapid method for inducing iPSC differentiation to obtain CD34+cells in this field.

NK cells are crucial for the body's defense and anti-tumor response, but the NK cells in tumor patients are usually functionally impaired. Therefore, the use of NK cells with normal or genetically modified functions to kill tumor cells, known as NK cell adoptive therapy, is currently the forefront and hotspot of cancer treatment. NK cell immunotherapy requires a large number of NK cells. Currently, the main sources of NK cells are: \textcircled{1} NK cells isolated from autologous/allogeneic peripheral blood (PB-NK), \textcircled{2} NK cells isolated from autologous/allogeneic umbilical cord blood (UCB-NK), \textcircled{3} NK cells derived from embryonic stem cell differentiation/induction of pluripotent stem cell differentiation (hESC-NK/iPSC-NK), and 4) NK cell lines such as NK-92. NK cells isolated from autologous peripheral blood are easily inhibited by the patient's own HLA molecules, which weakens the cell's killing ability, and it is usually difficult to isolate enough NK cells from the patient's body for clinical treatment. Although allogeneic peripheral blood can provide a large number of NK cells, there are significant differences in the number and cytotoxicity of NK cells isolated due to different donors. And PB-NK cells are not easily genetically modified. UCB-NK is not easily amplified, and UCB-NK is immature and has weak lethality. The NK-92 cell line exhibits polyploidy, uncontrolled proliferation, and potential tumorigenicity. And iPSC can differentiate into uniform, genotypic, and functionally similar NK cells to PB-NK, eliminating the donor differences present in PB-NK, which has important clinical application prospects.

There are currently three main methods for differentiating iPSC into NK cells: \textcircled{1} Co culturing iPSC with stromal cells to differentiate into hematopoietic progenitor cells, isolating hematopoictic progenitor cells, co culturing hematopoietic progenitor cells with stromal cells, and differentiating into NK cells. The entire differentiation process takes 47 to 55 days. \textcircled{2} Spreading iPSC onto a 96 well plate to form EB cells, differentiating into hematopoietic progenitor cells, and then transferring the EB cells into a 24 well or 6 well plate to differentiate into NK cells. The entire differentiation process takes 27 to 46 days, \textcircled{3} Placing iPSC single cells into a culture dish, wait for the iPSC clone to grow to the appropriate size, adding growth factors to stimulate cell differentiation to form hematopoietic progenitor cells, isolating hematopoietic progenitor cells, adding growth factors to stimulate NK cell differentiation, to obtain NK cells. The entire differentiation process takes 48 days. Among them, method \textcircled{1} requires co cultivation of iPSC with animal derived stromal cells, and the addition of animal derived components such as FBS during differentiation, making it unsuitable for clinical treatment. Method \textcircled{2}: the differentiation process is cumbersome and requires the addition of human serum, which is not conducive to large-scale preparation. Although the differentiation process is simple and there are no serum or animal derived components, which meets the clinical preparation conditions, the NK cells obtained have weaker cytotoxicity than PB-NK. It can be seen that the current methods of inducing NK cells by iPSC generally have the following shortcomings: low production of NK cells, poor cytotoxicity of differentiated NK cells, etc. How to overcome the aforementioned shortcomings of existing technologies and develop a new method for inducing differentiation of NK cells in vitro with good tumor killing activity is an urgent problem to be solved in the biological field.

### SUMMARY

The aim of the present invention is to provide a method and an application for inducing iPSC differentiation to obtain CD34+cells and NK cells, in response to the shortcomings of existing technologies. The method adopts an induction culture method of normoxia+hypoxia, which can start producing a high proportion of CD34+cells on the 4th day, and is significantly superior to the culture methods of normoxia+normoxia, hypoxia+hypoxia, and hypoxia+normoxia, significantly increasing the yield of iPSC induced NK cell differentiation, one iPSC can obtain approximately 10,000 NK cells, and the obtained NK cells have good killing function.

The above aim of the present invention is achieved through the following technical solutions:

The first aspect of the present invention provides a culture medium for inducing iPSC differentiation to obtain CD34+cells.

Furthermore, the culture medium comprises a first stage culture medium, a second stage culture medium, a third stage culture medium, and a fourth stage culture medium;
the first stage culture medium is an E8 complete culture medium containing ROCK pathway inhibitors and polyvinyl alcohol;
the second stage culture medium is an E8 complete culture medium containing GSK-3B inhibitors;
the third stage culture medium comprises SPM1 culture medium and SPM2 culture medium;
the fourth stage culture medium is SPM3 culture medium;
the SPM1 culture medium comprises stepro-34 complete culture medium, DMEM/F12 culture medium, L-glutamine, ascorbic acid, ITS-X, BMP4, VEGF, bFGF;
the SPM2 culture medium comprises SPM1 culture medium and the inhibitor of TGF-β type I receptors ALK5, ALK4, and ALK7;
the SPM3 culture medium comprises stepro-34 complete culture medium, DMEM/F12 culture medium, L-glutamine, ascorbic acid, ITS-X, bFGF, VEGF, SCF, TPO, and FLT-3L.

Furthermore, the ROCK pathway inhibitor in the first stage culture medium is Y-27632;
the GSK-3B inhibitor in the second stage culture medium is CHIR-99021;
the inhibitor of TGF-β type I receptors ALK5, ALK4, and ALK7 is SB431542;
the concentration of Y-27632 in the first stage culture medium is 0.5-20 μM;
the concentration of polyvinyl alcohol in the first stage culture medium is 2-6 mg/ml;
the concentration of CHIR-99021 in the second stage culture medium is 1-20 μM;
the concentrations of L-glutamine, ascorbic acid, ITS-X, BMP4, VEGF, bFGF, and SB431542 in the third stage culture medium are (0.1-5) % and (10-100) μg/mL, (0.1-5) ×. (10-100) ng/ml, (10-100) ng/ml, (10-100) ng/ml, (1-10) μM, respectively;
the concentrations of L-glutamine, ascorbic acid, ITS-X, bFGF, VEGF, SCF, TPO, and FLT-3L in the fourth stage culture medium are (0.1-5) %, (10-100) μg/mL, (0.1-5) ×, (10-100) ng/mL, (10-100) ng/mL, (10-100) ng/mL, (10-100) ng/mL, (1-50) ng/ml, respectively.

Furthermore, the concentration of Y-27632 in the first stage culture medium is 10 μM;
the concentration of polyvinyl alcohol in the first stage culture medium is 4 mg/mL;
the concentration of CHIR-99021 in the second stage culture medium is 10 μM;
the concentrations of L-glutamine, ascorbic acid, ITS-X, BMP4, VEGF, bFGF, and SB431542 in the third stage culture medium are 1%, 50% μg/mL, 1×, 50 ng/mL, 50 ng/mL, 50 ng/ml, 6 μM, respectively;

The concentrations of L-glutamine, ascorbic acid, ITS-X, bFGF, VEGF, SCF, TPO, and FLT-3L in the fourth stage culture medium are 1%, 50% μg/mL, 1×, 50 ng/ml, 50 ng/ml, 50 ng/ml, 30 ng/mL, 10 ng/ml, respectively.

The second aspect of the present invention provides a culture medium for inducing iPSC differentiation to obtain NK cells.

Furthermore, the culture medium comprises a first stage culture medium, a second stage culture medium, a third stage culture medium, a fourth stage culture medium, and a fifth stage culture medium;
the first stage culture medium, the second stage culture medium, the third stage culture medium, and the fourth stage culture medium are the first stage culture medium, the second stage culture medium, the third stage culture medium, and the fourth stage culture medium described in the first aspect of the present invention;
the fifth stage culture medium is SPM-NK culture medium;
the SPM-NK culture medium comprises stepro-34 complete culture medium, DMEM/F12 culture medium, L-glutamine, ascorbic acid, ITS-X, SCF, Flt-3L, IL-3, IL-7, and IL-15;
the concentrations of L-glutamine, ascorbic acid, ITS-X, SCF, Flt-3L, IL-3, IL-7, and IL-15 in the SPM-NK culture medium are (0.1-5) %, (10-100) μg/mL, (0.1-5) ×, (10-50) ng/ml, (1-20) ng/ml, (1-10) ng/mL, (10-50) ng/mL, (1-100) ng/mL, respectively.

Furthermore, the concentrations of L-glutamine, ascorbic acid, ITS-X, SCF, Flt-3L, IL-3, IL-7, and IL-15 in the SPM-NK culture medium are 1%, 50% μg/mL, 1×, 20 ng/ml, 10 ng/mL, 5 ng/mL, 20 ng/mL, 50 ng/mL, respectively.

The third aspect of the present invention provides a method for inducing iPSC to differentiate into CD34+cells.

Furthermore, the method comprises the following steps:
(1) a first stage, Day 1, under normoxic conditions, the iPSC is suspended and cultured using the first stage culture medium described in the first aspect of the present invention to form an embryoid bodies;
(2) a second stage, Day 0, under hypoxic conditions, the embryoid bodies are induced and cultured using the second stage culture medium described in the first aspect of the present invention to form mesodermal cells;
(3) a third stage, Day 1-Day 4, under hypoxic conditions, the mesodermal cells are induced and cultured using the third stage culture medium described in the first aspect of the present invention to form CD34+ hematopoietic endothelial cells;
(4) a fourth stage, Day 5-Day 12, the CD34+ hematopoietic endothelial cells were induced and cultured using the fourth stage culture medium described in the first aspect of the present invention under normoxic conditions to form CD34+/CD45+cells.

Furthermore, the first stage is iPSC induced differentiation to form embryoid bodies, the second stage is embryoid bodies induced differentiation to form mesodermal cells, the third stage is mesodermal cells induced differentiation to form CD34+ hematopoietic endothelial cells, and the fourth stage is CD34+ hematopoietic endothelial cells induced differentiation to form CD34+/CD45+cells.

Furthermore, the formation of embryoid bodies as described in step (1) includes the following steps: Day 1, digesting iPSC to a single cell state, inoculating cells, adding the first stage culture medium for resuspension culture, and forming embryoid bodies;

The density of the inoculated cells is 1×10⁵-2×10⁵/mL; The formation of CD34+ hematopoietic endothelial cells as described in step (3) includes the following steps:
(a) Day 1, inducing and culturing the mesodermal cells in SPM1 medium;
(b) Day 2, replacing the SPM1 medium with SPM2 medium for induction culture;
(c) Day 3, changing half of the solution, discarding half of the old SPM2 medium, and adding half of the new SPM2 medium;
(d) Day 4, adherent culturing embryonic bodies to form CD34+ hematopoietic endothelial cells.

Furthermore, the density of inoculated cells in step (1) is 1×10⁵/mL;

The cultivation conditions mentioned in step (1) are 5% CO₂ and 37° C. constant temperature cultivation;

The cultivation conditions mentioned in step (2) are 5% CO₂, 90% N2, and 37° C. constant temperature cultivation;

The cultivation conditions mentioned in step (3) are 5% CO₂, 90% N2, and 37° C. constant temperature cultivation;

The cultivation conditions mentioned in step (4) are 5% CO₂ and 37° C. constant temperature cultivation.

The fourth aspect of the present invention provides a method for inducing iPSC to differentiate into NK cells.

Furthermore, the method comprises the following steps based on the method described in the third aspect of the present invention: in the fifth stage, from Day 13 to Day 40, the CD34+/CD45+cells are induced and cultured using the fifth stage culture medium described in the second aspect of the present invention under normoxic conditions to form NK cells.

Furthermore, the fifth stage is to induce the differentiation of CD34+/CD45+cells into NK cells.

Furthermore, the fifth stage includes the following steps:
a) Day 13-Day 18, suspension culturing the CD34+/CD45+cells in the fifth stage culture medium;
b) Day 19-Day 40, replacing the fifth stage culture medium with a fifth stage culture medium without IL-3 for suspension culture to form NK cells;
The conditions for cultivation are 5% CO₂ and constant temperature cultivation at 37° C.

In the specific technical solution of the present invention, the three-dimensional structure of the embryoid body is utilized to provide a favorable differentiation microenvironment for iPSC differentiation. By forming the embryoid bodies, a high proportion of CD34+cells can be produced on the 4th day under hypoxic induction culture conditions. Subsequently, the production of iPSC-induced NK cell differentiation is significantly increased by adhering to the embryoid body wall or digesting and then suspending the embryoid body to induce differentiation. In the end, approximately 10,000 NK cells can be obtained from one iPSC, and the obtained NK cells have good killing function against tumor cells.

The fifth aspect of the present invention provides a CD34+cell population or a derivative thereof, or an NK cell population or a derivative thereof.

Furthermore, the CD34+cell population is induced and differentiated using the method described in the third aspect of the present invention, and the NK cell population is induced and differentiated using the method described in the fourth aspect of the present invention; The CD34+cell population simultaneously expresses CD45;

The derivative of the CD34+cell population is a hematopoietic cell line cell population obtained by inducing differentiation of the CD34+cell population;

The hematopoietic cell line cell population induced by differentiation of the CD34+cell population includes T cells, NK cells, B cells, and macrophages.

The sixth aspect of the present invention provides a pharmaceutical composition for treating and/or preventing hematological and/or autoimmune diseases and/or solid tumors.

Furthermore, the pharmaceutical composition comprises a CD34+cell population or a derivative thereof, an NK cell population or a derivative thereof as described in the fifth aspect of the present invention;

Preferably, the pharmaceutical composition also comprises pharmaceutically acceptable carriers and/or excipients;

Preferably, the hematological system diseases include chronic myeloid leukemia, acute myeloid leukemia, acute lymphocytic leukemia, non Hodgkin's lymphoma, Hodgkin's lymphoma, multiple myeloma, myelodysplastic syndrome, aplastic anemia, Fanconi's anemia, thalassemia, sickle cell anemia, bone marrow fibrosis, severe paroxysmal nocturnal hemoglobinuria Non megakaryocytic thrombocytopenia;

Preferably, the autoimmune diseases include refractory rheumatoid arthritis, systemic lupus erythematosus, multiple sclerosis, juvenile idiopathic arthritis, systemic sclerosis, Wegener granuloma, antiphospholipid antibody syndrome, severe myasthenia gravis, Crohn's disease, type I diabetes, and severe combined immunodeficiency;

Preferably, the solid tumors include breast cancer, ovarian cancer, testicular cancer, neuroblastoma, small cell lung cancer, nasopharyngeal cancer, retroperitoneal yolk sac tumor, Ewing sarcoma, primitive neuroectodermal tumor, nephroblastoma, liver cancer, malignant schwannoma, and retinoblastoma.

Furthermore, the pharmaceutically acceptable carriers and/or excipients are detailed in Remington's Pharmaceutical Sciences (19th ed., 1995), and these substances are used as needed to aid in the stability of the formula, enhance its activity or bioavailability, or produce an acceptable taste or odor when taken orally. The formulations that can be used in this pharmaceutical composition can be in the form of their original compound itself; alternatively, use the pharmaceutically acceptable form of salt. Preferably, the pharmaceutically acceptable carriers and/or excipients include pharmaceutically acceptable carriers, diluents, fillers, binders, and other excipients, depending on the administration method and the designed dosage form. Preferably, the pharmaceutical composition is any pharmaceutically acceptable dosage form, including at least one of tablets, capsules, injections, granules, suspensions, and solutions. Preferably, the suitable dosage of the drug composition can be prescribed in multiple ways based on factors such as formulation method, administration method, patient's age, weight, gender, pathology, diet, administration time, administration route, excretion rate, and reaction sensitivity. Skilled doctors can usually easily determine the effective dosage of the prescription and prescription for the desired treatment.

Furthermore, the actual dose of the active ingredient in the pharmaceutical composition (CD34+cell population or its derivatives, NK cell population or its derivatives as described in the fifth aspect of the present invention) should be determined based on various relevant factors, including the severity of the disease to be treated, the course of use, patient age, gender, and body weight. Therefore, the above dose should not in any way limit the scope of protection of the present invention.

The seventh aspect of the present invention provides applications in any of the following aspects:
(1) application of the culture medium described in the first aspect of the present invention in inducing iPSC differentiation to obtain CD34+cells;
(2) application of the culture medium described in the second aspect of the present invention in inducing iPSC differentiation to obtain NK cells;
(3) application of CD34+cell populations or their derivatives, NK cell populations or their derivatives in the preparation of drugs for the treatment and/or prevention of hematological and/or autoimmune diseases and/or solid tumors, as described in the fifth aspect of the present invention;
Preferably, the hematological system diseases include chronic myeloid leukemia, acute myeloid leukemia, acute lymphocytic leukemia, non Hodgkin's lymphoma, Hodgkin's lymphoma, multiple myeloma, myelodysplastic syndrome, aplastic anemia, Fanconi's anemia, thalassemia, sickle cell anemia, bone marrow fibrosis, severe paroxysmal nocturnal hemoglobinuria, and megakaryocytic thrombocytopenia;
Preferably, the autoimmune diseases include refractory rheumatoid arthritis, systemic lupus erythematosus, multiple sclerosis, juvenile idiopathic arthritis, systemic sclerosis, Wegener granuloma, antiphospholipid antibody syndrome, severe myasthenia gravis, Crohn's disease, type I diabetes, and severe combined immunodeficiency;
Preferably, the solid tumors include breast cancer, ovarian cancer, testicular cancer, neuroblastoma, small cell lung cancer, nasopharyngeal cancer, retroperitoneal yolk sac tumor, Ewing sarcoma, primitive neuroectodermal tumor, nephroblastoma, liver cancer, malignant schwannoma, and retinoblastoma.

In order to further explain the present invention, some professional terms involved in the present invention are explained as follows:

The term “hematopoietic stem cells (HSCs)” mentioned in the present invention refers to immature blood cells that have the ability to self renew and differentiate into more mature blood cells. The more mature blood cells include granulocytes (such as premyelocytes, neutrophils, cosinophils, basophils), red blood cells (such as reticulocytes, red blood cells) Coagulation cells (such as megakaryocytes, platelet producing megakaryocytes, platelets) and monocytes (such as monocytes, macrophages). In the specification, HSCs are interchangeably referred to as stem cells. It is known in this field that such cells may or may not include CD34+cells. CD34+cells are immature cells that express surface markers of CD34 cells. It is believed that CD34+cells include subpopulations of cells with stem cell properties as defined above. As is well known, HSCs include pluripotent stem cells, pluripotent stem cells (such as lymphoid stem cells), and/or stem cells classified as special hematopoietic cell lines. Stem cells classified as special hematopoietic cell lines can be T cell lines, B cell lines, dendritic cell lines, Langerhans cell lines, and/or lymphoid tissue-specific macrophage lines. In addition, HSC also involves long-term HSC (LT-HSC) and short-term HSC (ST-HSC). ST-HSC has higher vitality and stronger proliferation than LT-HSC. However, LT-HSC has unrestricted self-renewal (i.e., its survival lasts throughout adulthood), while ST-HSC has limited self-renewal (i.e., it only survives for a limited period of time). Any of these HSCs can be used in any of the methods described in the present invention. Optionally, ST-HSC is useful due to their high proliferative properties and the ability to rapidly increase the number of HSCs and their offspring. Hematopoietic stem cells are optionally derived from blood products. A blood product includes products derived from the body or bodily organs containing hematopoietic derived cells. This type of source includes unrated bone marrow, umbilical cord, peripheral blood, liver, thymus, lymph, and spleen. The crude or unclassified blood products mentioned above can be enriched with cells with hematopoietic stem cell characteristics in a way known to those skilled in the art.

The term “embryoid body (EB)” referred to in the present invention refers to a homogeneous or heterogeneous cell cluster consisting of differentiated cells, partially differentiated cells, and/or pluripotent stem cells cultured in suspension. In order to summarize some inherent clues of differentiation in the body, the present invention uses three-dimensional embryoid bodies as intermediate steps. At the beginning of cell aggregation, differentiation can be initiated and cells can begin to reproduce embryonic development to a limited extent. Although they cannot form nourishing ectodermal tissue, almost all other types of cells present in organisms can develop. The present invention can further promote the differentiation of hematopoietic progenitor cells after the formation of embryonic bodies.

The term “treatment and/or prevention” referred to in the present invention refers to the prevention, reversal, alleviation, or inhibition of the disorder or disease to which the term applies, or the progression of one or more symptoms of such disorder or disease. The treatment of a disease or condition includes the improvement of at least one symptom of a specific disease or condition, even if the underlying pathophysiology is not affected, for example, The term ‘treating and/or preventing hematological diseases’ used in the present invention includes one or more of the following: (1) preventing the occurrence of hematological diseases; (2) Inhibiting the development of hematological diseases; (3) Curing blood system diseases; (4) Relieving symptoms related to patients with hematological diseases; (5) Reducing the severity of blood system diseases; (6) Preventing the recurrence of blood system diseases.

The term “human induced pluripotent stem cells” referred to in the present invention, also known as “induced pluripotent stem cells”, is commonly abbreviated as iPS cells or iPSC. It refers to a type of pluripotent stem cell that is artificially prepared from non pluripotent cells (usually adult cells) or terminally differentiated cells (such as fibroblasts, hematopoietic cells) by introducing or contacting reprogramming factors, such as muscle cells, neurons, epidermal cells, etc.

Compared to the prior art, the present invention has the following advantages and beneficial effects:

The existing methods for inducing iPSC directed CD34+cell differentiation (such as embryoid differentiation method and adherent differentiation method) have the disadvantages of long induction time and generally low yield. Compared to the existing technologies mentioned above, the present invention provides a culture medium combination and preparation method for inducing pluripotent stem cell differentiation to obtain CD34+cells and NK cells, and the culture medium combination and preparation method. Under hypoxia induced culture conditions, a high proportion of CD34+cells can begin to be produced on the 4th day. Subsequently, the production of iPSC induced NK cell differentiation was significantly increased through EB adhesion or digestion followed by suspension induction. The induced NK cells can also exert a killing effect on tumor cells in a short period of time, exhibiting strong tumor killing ability. The method provided by the present invention significantly overcomes the problems of low NK cell yield and poor NK cell killing function in existing NK cell differentiation techniques, and is suitable for the production and clinical application of large-scale cell preparations. In addition, the present invention for the first time found that in inducing iPSC to differentiate into CD34+cells, the induction culture method of normoxia+hypoxia is significantly superior to the induction culture method of normoxia+normoxia, hypoxia+hypoxia, and hypoxia+normoxia, achieving unexpected technical effects.

### BRIEF DESCRIPTION OF DRAWINGS

FIG. 1 is the experimental flowchart of inducing iPSC differentiation to obtain NK cells according to the present invention;

FIG. 2 shows the cell morphology of iPS cells under a 4× optical microscope;

FIG. 3 shows the cell morphology of Day 0, Day 4, Day 5, Day 12, Day 26, and Day 40 cells during the differentiation process under a 4× optical microscope;

FIG. 4 shows the morphological results of NK cells observed under a 20× optical microscope on the 40th day of differentiation;

FIG. 5 shows the detection results of CD34, CD31, and CD235 expression on the 4th day of differentiation. Figure A shows CD34, CD31, and Figure B shows CD34, CD235;

FIG. 6 shows the detection results of CD34 and CD45 expression on the 12th day of differentiation;

FIG. 7 shows the detection results of CD122, CD45, and CD56 expression on the 26th day of differentiation. Figure A shows CD122, CD45, and Figure B shows CD122, CD56;

FIG. 8 shows the detection results of CD45, CD56, and CD3 positive cell expression on the 40th day of differentiation. Figure A shows CD45 and CD56, while Figure B shows CD3 and CD56;

FIG. 9 shows the results of NK cell related maker detection on the 40th day of differentiation, with Figure A showing Granzyme B and Figure B showing NKP46;

FIG. 10 shows the results of the NK cell killing function validation experiment obtained through induced differentiation, where Figure A shows the LNCap cell line and Figure B shows the K562 cell line;

FIG. 11 shows the detection results of CD34 and CD235 expression under culture conditions of normoxia+hypoxia, normoxia+normoxia, hypoxia+normoxia, and hypoxia+hypoxia on the 4th day of differentiation. Among them, Figure A shows normoxia+hypoxia (normoxia during embryoid formation, hypoxia during mesoderm and endothelial formation), and Figure B shows normoxia+normoxia (normoxia during embryoid formation, mesoderm, and endothelial formation), Figure C: Hypoxia+normoxia (hypoxia is present in the stages of embryonic body formation, mesoderm, and endothelial cell formation), Figure D: Hypoxia+hypoxia (hypoxia is present in the stages of embryonic body formation, mesoderm, and endothelial cell formation);

FIG. 12 shows the detection results of CD34 and CD45 expression in suspension cells under conditions of normoxic+hypoxia, normoxic+normoxic, hypoxic+normoxic, and hypoxic+hypoxic culture on the 12th day of differentiation. Among them, Figure A shows normoxic+hypoxia, Figure B shows normoxic+normoxic, Figure C shows hypoxia+normoxic, and Figure D shows hypoxia+hypoxia;

FIG. 13 shows the detection results of CD56 and CD45 expression in suspended cells under culture conditions of normoxia+hypoxia, normoxia+normoxia, hypoxia+normoxia, and hypoxia+hypoxia on the 40th day of differentiation. Among them, Figure A shows normoxia+hypoxia, Figure B shows normoxia+normoxia, Figure C shows hypoxia+normoxia, and Figure D shows hypoxia+hypoxia;

FIG. 14 shows the expression of CD34 and CD43 under different initial cell densities on the 12th day of differentiation;

FIG. 15 shows the expression of CD34 at different bFGF concentrations detected by flow cytometry on the 4th day of differentiation;

FIG. 16 shows the results of flow cytometry detection of CD34 expression at different concentrations of SB431542 on the 4th day of differentiation. Figure A shows the results of flow cytometry detection of CD34 expression on the 6th day of differentiation, while Figure B shows the results of flow cytometry detection of CD34 expression on the 12th day of differentiation;

FIG. 17 shows the results of detecting the expression of biomarkers in cord blood derived NK and iNK. Figure A shows the expression of CD56, CD45, and CD3 in cord blood derived NK and iPSC induced differentiation derived NK by flow cytometry, while Figure B shows the expression of Granzyme B, CD94, NKP30, NKP44, NKP46, and TRAIL in cord blood derived NK and iPSC induced differentiation derived NK by flow cytometry, Figure C: Flow cytometry detection of Gramzyme B and IFN induced differentiation of NK from umbilical cord blood and three different differentiation batches of iPSC-γ, statistical results of CD94, NKG2D, NKP30, NKP44, NKP46, and TRAIL expression;

FIG. 18 shows the comparison of tumor killing ability between NK cells derived from cord blood and iNK cells prepared by the present invention.

### DETAILED DESCRIPTION

The following will further elaborate on the present invention in conjunction with specific embodiments, which is only intended to explain the present invention and cannot be understood as a limitation of the present invention. Ordinary technical personnel in this field can understand that various changes, modifications, substitutions, and variations can be made to these embodiments without departing from the principles and purposes of the present invention. The scope of the present invention is limited by the claims and their equivalents. The experimental methods used in the following embodiments are conventional methods unless otherwise specified; The reagents, biomaterials, etc. used in the following embodiments can be obtained commercially unless otherwise specified.

### Example 1 Experimental Process of Inducing iPSC Differentiation to Obtain CD34+Cells and NK Cells

### 1. Experimental Materials

The experimental materials involved in the examples of the present invention are shown in Table 1.

TABLE 1
Experimental materials

| Experimental materials name | Manufacturer | Article number |
|---|---|---|
| matrigel | Corning | 354277 |
| StemPro-34 SFM | Thermo | 10640-019 |
| StemPro-34 Nutrient | Thermo | 10641-025 |
| DMEM/F-12 with HEPES | Thermo | 11330-032 |
| GlutaMAX-I(100X) | Gibco | 35050061 |
| Insulin-Transferrin-Selenium-X | Gibco | 51500056 |
| L-ascorbic acid | sigma | A92902 |
| BMP4 | peprotech | 120-05ET |
| Animal-Free Recombinant | peprotech | AF-100-18B |
| Human FGF-basic(154 a.a.) | | |
| Animal-Free Recombinant | peprotech | AF-100-20 |
| Human VEGF165 | | |
| CHIR99021 | StemCellTechnologies | 72054 |
| SB431542 | abcam | ab120163 |
| 0.25% Trypsin-EDTA(1X) | Thermo | 25200072 |
| E8 Basal Medium | STEMCELL | 05991 |
| E8 25X Supplement | STEMCELL | 05992 |
| Recombinant Human TPO | peprotech | 300-18-10 |
| Recombinant Human SCF | peprotech | 300-07 |
| Animal-Free Recombinant | peprotech | AF-300-19 |
| Human Flt3-Ligand | | |
| IL-7 | peprotech | 200-07 |
| Recombinant Human IL-15 | peprotech | 200-15 |
| Recombinant Human IL-21 | peprotech | 200-21 |
| IL-3 | peprotech | 200-03-10 |
| Human IL-2 | peprotech | 200-02 |

### 2. Formation of Embryoid Bodies (EB)

(1) IPSC originated from ALLIFE MEDICINE (BEIJING) LIMITED and was prepared using the method described in our company's previously filed patent application (201910110768.7). After the growth convergence of iPSC reached 70%, the supernatant was aspirated, and pre preheated DPBS was added to clean the cells twice. Then, pre preheated Tryple Express was added to digest the cells in a single cell state. After digestion and centrifugation were terminated, the supernatant was removed, and the cells were resuspension cultured by using E8 medium containing 10 μM ROCK pathway inhibitor Y-27632+E8 complete medium containing 4 mg/mL polyvinyl alcohol (PVA), for cell counting;

(2) Adjusting cell density to 1×10⁵−2×10⁵/mL, inoculating cells into a low absorption six well plate for suspension culture, with 3 mL of culture medium per well, i.e. 50-600,000 cells per well, and culture cells in a 5% CO₂, 37° C. constant temperature incubator for 24 hours, labeled as Day-1, to form EB (see Example 4 below for experimental results of different cell densities);

In this example, the ROCK pathway inhibitor is 0.5-20 μM Y-27632; Small molecule substances that can perform similar functions include but are not limited to: Thiazovivin, Fasudil (HA-1077) HCl, GSK429286A, RKI-1447, Azaindole 1.

3. Inducing the Initiation of Embryoid Differentiation into the Mesoderm

Transferring EB to a centrifuge tube, centrifuging 20 g for 2 minutes, removing the supernatant, and adding E8 complete culture medium containing 10 μM GSK-3 β Inhibitor CHIR-99021, initiated mesodermal differentiation, marked as Day 0, and cells were initially cultured in a constant temperature incubator at 5% CO₂, 90% N2, and 37° C. for 24 hours;

In this example, GSK-3 B Inhibitor is 1-10 μM CHIR-99021; small molecule substances that can perform similar functions include but are not limited to: SB216763, CHIR-98014, TWS119, Tideglusib, SB415286.

In this example, the differentiation induction basic culture medium includes but is not limited to: E8 complete culture medium, StemPro-34, and Stemline® II. STEMdiff™ APEL™ 2 Medium.

4. Inducing Mesodermal Cells to Differentiate into CD34+Hematopoietic Endothelial Cells (HECs)
(1) Day 1, replaced with SPM1 medium, which consists of 50% stepro-34 complete medium, 50% DMEM/F12 medium, 1% L-glutamine, and 50 μg/mL ascorbic acid, 1 ×Insulin-Transferrin-Selenium-Ethanolamine (ITS-X), 50 ng/mL BMP4, 50 ng/mL VEGF, and 50 ng/mL bFGF, the EB of Day 0 was resuspended in SPM1 medium and cultured in a constant temperature incubator at 5% CO₂, 90% N2, and 37° C. for 24 hours (experimental results of different bFGF concentrations are shown in Example 5 below);
(2) Day 2, replaced with SPM2 medium, which on the basis of SPM1, adding 6 μM of inhibitor SB431542 of TGF-β Inhibitors type I receptors ALK5, ALK4, and ALK7, which was added to 3 mL per well. The EB of Day 1 was resuspended in SPM2 medium and cultured in a constant temperature incubator at 5% CO₂, 90% N2, and 37° C. for 24 hours (see Example 6 for the comparative experimental results with and without addition of SB431542);
(3) Day 3, changed half of the solution, discarded half of the old SPM2 medium, and added half of the new SPM2 medium;
(4) Day 4, collected a portion of EB for flow detection, and the detection method is shown in Example 2;

In this example, the inhibitors of TGF-β type I receptors ALK5, ALK4, and ALK7 are 1-6 μM of SB431542, small molecule substances that can perform similar functions include but are not limited to: Galunisertib (LY2157299), LY2109761, SB525334, SB505124, GW788388.

5. Inducing CD34+ Hematopoietic Endothelial Cells to Differentiate into CD34+/CD45+ hematopoietic stem cells (HSCs)
(1) Day 5, transferred EB to Matrigel pre coated cell culture dish, while replaced SMP3 medium. SPM3 medium consisted of 50% stepro-34 complete medium, 50% DMEM/F12 medium, 1% L-glutamine, and 50% μg/mL ascorbic acid, 1×Insulin-Transferrin-Selenium-Ethanolamine (ITS-X), 50 ng/mL bFGF, 50 ng/mL VEGF, 50 ng/mL SCF, 30 ng/mLTPO, and 10 ng/mLFLT-3L, the cells were cultured in a 5% CO₂, 37° C. constant temperature incubator until Day 12. During this period, the solution was changed every three and a half days to obtain CD34+/CD45+suspension cells.

In this example, the culture dish coating matrix includes but is not limited to: Mtrigel, Geltin, Lamin521, or Fibronection.

(2) Day 12, collected suspended cells for flow cytometry detection, and performed flow cytometry detection on the suspended cells. The detection method is shown in Example 2.

In this example, the E8 culture medium can be a product of Stem Cell, the stepro-34, DMEM/F12 Medium, and TrypLE can all be products of Thermo, the BMP4, Human Recombinant VEGF165 (VEGFA), Human Recombinant SCF, Human Recombinant Flt-3L, Human Recombinant bFGF, and Human Recombinant TPO can all be products of Peprotech, and the Y-27632, CHIR99021, and SB431542 can all be products of Sigma, the matrigel mentioned can all be products of Corning Corporation.

6. Inducing CD34+/CD45+ Hematopoietic Stem Cells to Differentiate into NK Cells
(1) Day 13, collected the supernatant into a 50 mL centrifuge tube, centrifuged 250 g for 5 minutes, and removed the supernatant;
(2) Resuspension cells using SPM-NK complete culture medium, which consists of 50% stepro-34 complete culture, 50% DMEM/F12, 1% L-glutamine, and 50% μg/mL ascorbic acid, 1×Insulin-Transferrin-Selenium-Ethanolamine (ITS-X), 20 ng/mL SCF, 10 ng/mL Flt-3L, 5 ng/ml IL-3, 20 ng/ml IL-7, and 50 ng/ml IL-15 were cultured in a constant temperature incubator with 5% CO₂ at 37° C., and the solution was changed every two and a half days during this period;
(3) On the 19th day of differentiation, the suspended cells were collected into a 50 mL centrifuge tube and centrifuged at 250 g for 5 minutes. The supernatant was removed, and new SPM-NK medium without IL-3 was added to resuspend the cells. The cells were cultured in a 5% CO₂, 37° C. constant temperature incubator, and the medium was changed every two and a half days during this period;
(4) On the 26th day of differentiation, the supernatant was transferred to a new culture dish, and some suspension cells were collected for flow cytometry to detect the expression of CD45, CD122, and CD56;
(5) On the 40th day of differentiation, suspended cells were collected and the expression of CD45, CD56, CD3, NKP46, and Granzyme B was detected by flow cytometry. The detection method is shown in example 2; simultaneously collected some suspended cells and conducted NK cell killing experiments for verification. The specific experimental method is shown in example 3.

In this example, the E8 culture medium can be a product of Stem Cell, and the stepro-34, DMEM/F12 Medium, and TrypLE can all be products of Thermo. The BMP4, Human Recombinant VEGF165 (VEGFA), Human Recombinant SCF, Human Recombinant Flt-3L, Human Recombinant bFGF, Human Recombinant TPO, IL-3, IL-7, IL-15, and IL-21 can all be products of Peprotech. The Y-27632, CHIR99021 SB431542 can all be products of Sigma Company, and the matrigel can all be products of Corning Company.

### Example 2 Embryoid Bodies Flow Detection and Suspension Cell Flow Detection

1. Embryoid bodies flow detection

The specific experimental steps for embryoid bodies flow detection are as follows:
(1) Transferred the embryoid bodies to a 15 mL centrifuge tube, centrifuged 20 g for 2 minutes, and removed the supernatant;
(2) Added 1 mL of DPBS for cleaning, centrifuged 20 g for 2 minutes, and removed the supernatant;
(3) Added 1 mL of 0.25% trypsin and digested at 37° C. for 5 minutes, during which blowing once every 2 minutes;
(4) Added DPBS containing 4% FBS to terminate digestion, centrifuged 250 g for 5 minutes, and removed the supernatant;
(5) Added 1 mL of DPBS to clean the cells once;
(6) Resuspended cells by using 100 μL of DPBS containing 4% FBS;
(7) Added corresponding flow detection antibodies and incubated at 4° C. for 30 minutes;
(8) Centrifuged 250 g to remove the supernatant, and added 1 mL of DPBS to clean the cells 3 times;
(9) 200 μL DPBS was used to resuspend cells for testing.

### 2. Suspension Cell Flow Detection

The specific experimental steps for suspension cell flow detection are as follows:
(1) Transferred the supernatant into a 15 mL centrifuge tube, centrifuged 250 g for 5 minutes, and removed the supernatant;
(2) Added 1 mL of DPBS to clean the cells once;
(3) Resuspended cells by using 100 μL DPBS containing 4% FBS;
(4) Added corresponding flow detection antibodies and incubated at 4° C. for 30 minutes;
(5) Centrifuged 250 g to remove the supernatant, and added 1 mL of DPBS to clean the cells 3 times;
(6) Resuspended the cells by using 200 μL DPBS for testing.

### 3. Experimental Results

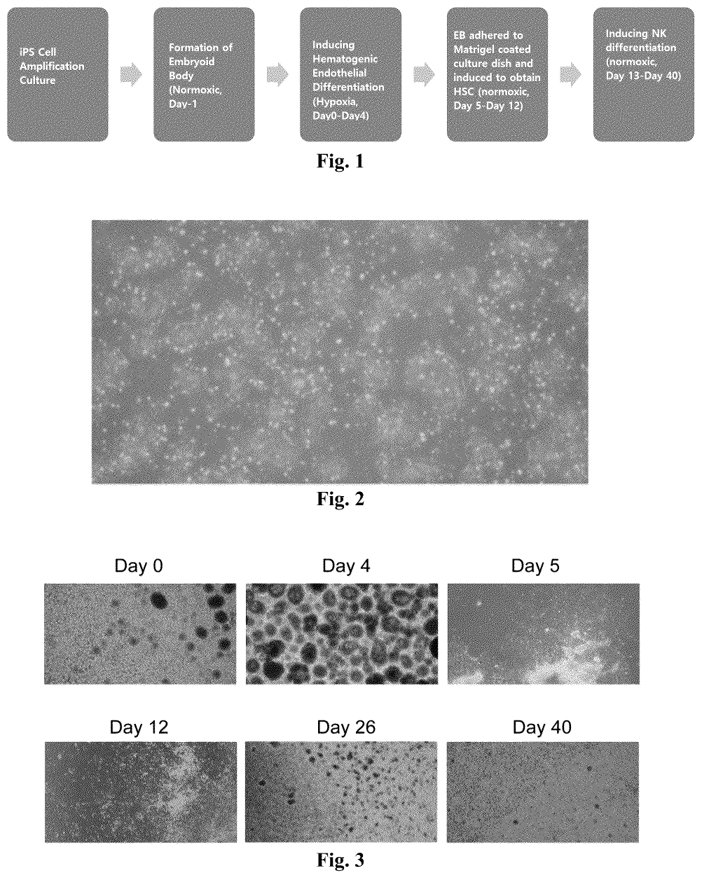

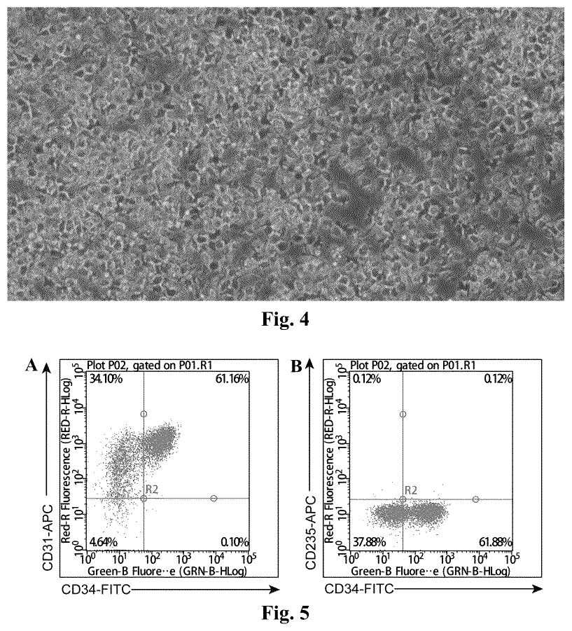

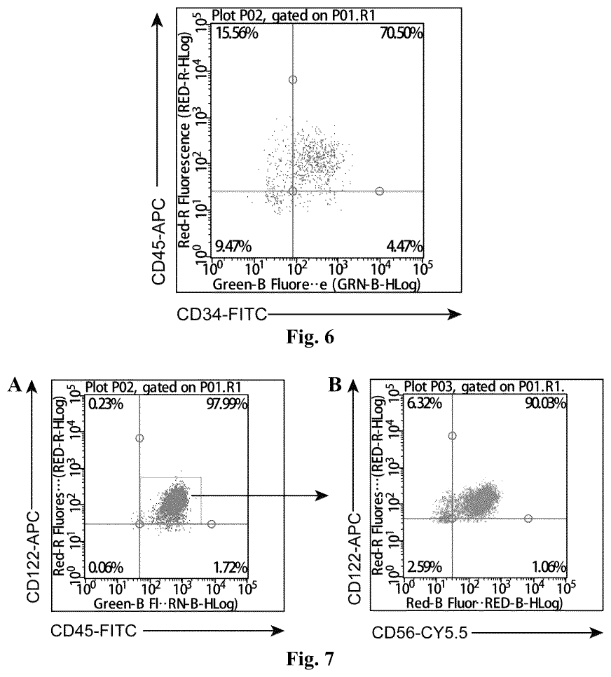

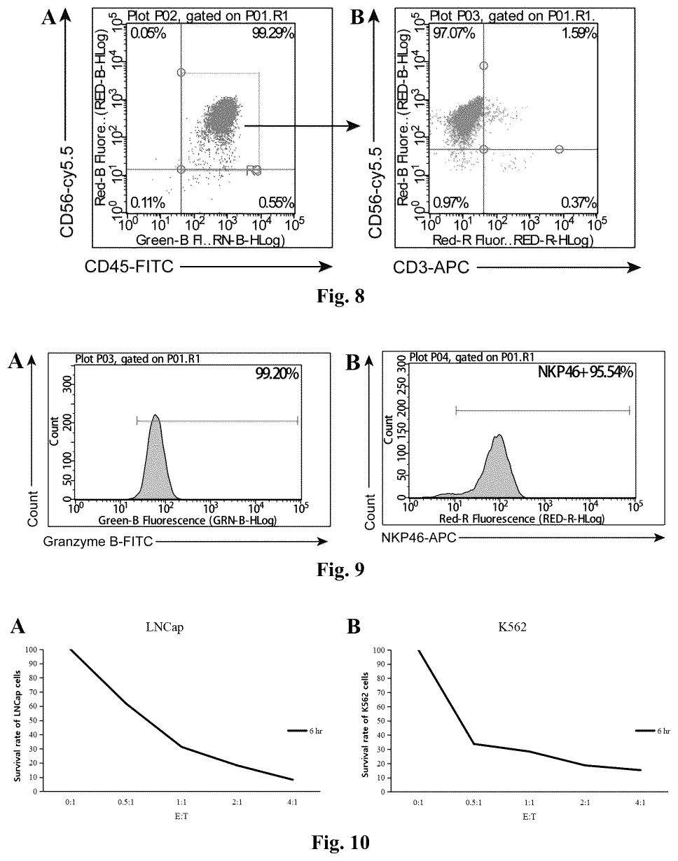

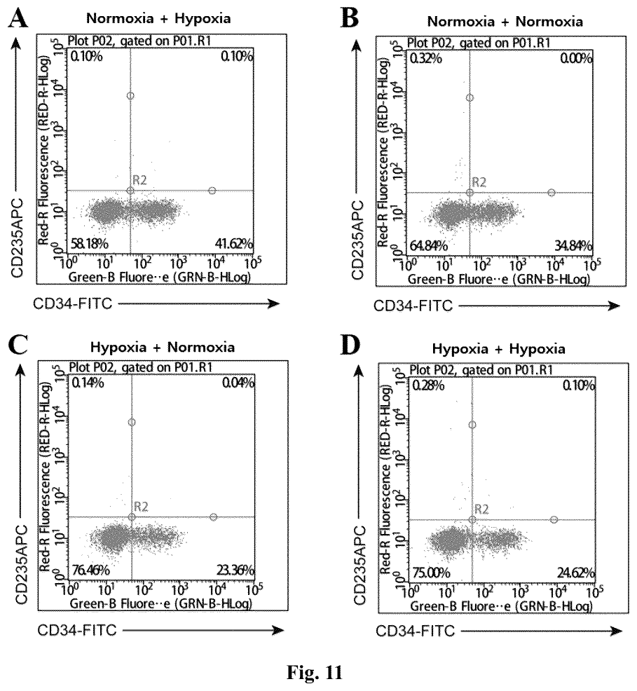

The experimental flowchart of inducing iPSC differentiation to obtain NK cells in the present invention is shown in FIG. 1, and the cell morphology of iPS cells under a 4× optical microscope is shown in FIG. 2. When the convergence degree of iPSC reaches 70%-80%, it begins to enter differentiation; during the differentiation process, the cell morphology of Day 0, Day 4, Day 5, Day 12, Day 26, and Day 40 cells under a 4× optical microscope is shown in FIG. 3. The results show that at the beginning of differentiation (Day 0), a smooth edged embryoid body is formed; on the 4th day of differentiation, the embryoid body increased and obvious cavities appeared; on the 5th day of differentiation, uniform hematopoietic endothelial like cells were extended around the embryonic body; suspended cells appeared on the 12th day of differentiation; on the 26th day of differentiation, the number of suspended cells significantly increased and hippocampal shaped NK cell morphology cells appeared; on the 40th day of differentiation, a large number of hippocampal like NK shaped cells appeared; on the 40th day of differentiation, the morphology of NK cells was observed under a 20× optical microscope, as shown in FIG. 4. The results showed that most of the suspended cells on the 40th day of differentiation exhibited a hippocampal like morphology of NK cells; on the 4th day of differentiation, the expression of CD34, CD31, and CD235 was detected as shown in FIG. 5. The results showed that at least more than 5% of CD34 positive cells and at least 5% of CD31 positive cells were obtained on the 4th day of differentiation, and the proportion of CD235 negative cells did not exceed 30%; the detection results of CD34 and CD45 expression on the 12th day of differentiation are shown in FIG. 6. The results show that at least 5% of CD34 and CD45 double positive cells were obtained on the 12th day of differentiation; the detection results of CD122, CD45, and CD56 expression on the 26th day of differentiation are shown in FIG. 7. The results show that at least 5% of CD45 and CD122 positive cells and at least 2% of CD56 positive cells were obtained on the 26th day of differentiation; the expression of CD45 and CD56 positive cells was detected on the 40th day of differentiation, as shown in FIG. 8. The results showed that at least 5% of CD45 and CD56 double positive cells were obtained on the 40th day of differentiation, while CD3 positive cells did not exceed 30%; the results of NK cell related maker detection on the 40th day of differentiation are shown in FIG. 9. The results show that the proportion of NKP46 positive cells on the 40th day of differentiation is at least 5%, and the proportion of Granzyme B positive cells is at least 5%.

### Example 3 Verification of NK cell killing function obtained through induced differentiation

1. Experimental method
(1) Collected target tumor cells and NK cells for cell counting. The target tumor cells include human prostate cancer LNCap cells and human chronic myeloid leukemia K562 cells (LNCap cells were purchased from Procell Company and K562 from Beina Biotechnology Co., Ltd.);
(2) Inoculated NK cells and tumor cells in a target ratio of 0:1, 0.5:1, 1:1, 2:1, and 4:1 into a 96 well plate, while setting up blank control wells and corresponding NK cell separate culture wells, and cultured them in a 5% CO₂, 37° C. constant temperature incubator;
(3) Added 15 μL Alamar Blue to each hole after 3 hours, cultured in a constant temperature incubator with 5% CO₂ at 37° C.;
(4) Performed enzyme-linked immunosorbent assay after 3 and 6 hours, respectively. 2. Experiment results

The results of the NK cell killing function validation experiment are shown in FIG. 10. The results show that after 6 hours of killing, the survival rates of LNCap and K562 cells decrease with the increase of ET ratio, indicating that the NK cells prepared by the method described in the present invention can exert a killing effect on tumor cells in a short period of time and have strong tumor killing ability.

### Example 4 Effects of Different Experimental Conditions on Inducing iPSC Differentiation to Produce CD34+Cells and NK Cells

### 1. The Formation of Embryoid Bodies (EB)

(1) After the convergence rate of iPSC (sourced from ALLIFE MEDICINE (BEIJING) LIMITED) reaches 70%, the supernatant was aspirated, and pre preheated DPBS was added to clean the cells twice. Then, pre preheated Tryple Express was added to digest the cells into a single cell state. After stopping digestion and centrifugation, the supernatant was removed, and the cells were resuspended by adding E8 medium containing 10% μM ROCK pathway inhibitor Y-27632+E8 complete medium containing 4 mg/mL polyvinyl alcohol (PVA), conducting cell counting;

(2) adjusted the cell density to 1.5×103-2×104/mL, inoculated cells into a low absorption six well plate for suspension culture, with 3 mL of culture medium per well, i.e. 50-600,000 cells per well. The A and B culture plates were placed in 5% CO₂ (under normoxic conditions), and the cells were cultured in a 37° C. constant temperature incubator for 24 hours. In addition, the C and D culture plates were placed in 5% CO₂, 90% N2 (under hypoxic conditions), and the cells were cultured in a 37° C. constant temperature incubator for 24 hours, marked as Day-1, forming EB;

In this example, the ROCK pathway inhibitor is 0.5-20 μM of Y-27632; small molecule substances that can perform similar functions include but are not limited to: Thiazovivin, Fasudil (HA-1077) HCl, GSK429286A, RKI-1447, Azaindole1.

2. Inducing the initiation of embryoid bodies differentiation into the mesoderm

Transferred four EBs with low absorption of 6-well plates to a centrifuge tube, centrifuged 20 g for 2 minutes, removed the supernatant, and added E8 complete culture medium containing 10 μM GSK-3B Inhibitor CHIR-99021 to initiate mesodermal differentiation, marked as Day 0. The A and D culture plates were placed in 5% CO₂, 90% N2 (hypoxic conditions), and the cells were cultured in a 37° C. constant temperature incubator for 24 hours. Incubated the B and C culture plates in a constant temperature incubator at 37° C. for 24 hours at 5% CO₂ (under normoxic conditions);

In this example, GSK-3B Inhibitors are 1-10 μM of CHIR-99021; small molecule substances that can perform similar functions include but are not limited to: SB216763, CHIR-98014, TWS119, Tideglusib, SB415286.

In this example, the differentiation induction basic culture medium includes but is not limited to: E8 complete culture medium, StemPro-34, and Stemline® II. STEMDiff™ APEL™ 2 Medium.

3. Inducing Mesodermal Cells to Differentiate into CD34+Hematopoietic Endothelial Cells (HECs)

(1) Day 1, replaced with SPM1 medium, which consists of 50% stepro-34 complete medium, 50% DMEM/F12 medium, 1% L-glutamine, and 50 μg/mL ascorbic acid, 1×Insulin-Transferrin-Selenium Ethanolamine (ITS-X), 50 ng/ml BMP4, 50 ng/mL VEGF, and 50 ng/mL bFGF. The EB of Day 0 was resuspended in SPM1 medium, and the A and D culture plates were initially placed in 5% CO₂, 90% N2 (hypoxic conditions), and incubated for 24 hours in a 37° C. constant temperature incubator. Incubated the B and C culture plates in a constant temperature incubator at 37° C. for 24 hours at 5% CO₂ (under normoxic conditions);

(2) Day 2, replaced with SPM2 medium, which on the basis of SPM1 adding 6 μM inhibitors SB431542 of TGF-βype I receptors ALK5, ALK4, and ALK7, which was added to 3 mL per well. The EB from Day 1 was resuspended in SPM2 medium, and the A and D plates were placed in a constant temperature incubator at 37° C. for 24 hours under 5% CO₂, 90% N2 (hypoxic conditions). Incubated the B and C culture plates in a constant temperature incubator at 37° C. for 24 hours at 5% CO₂ (under normoxic conditions);

(3) Day 3, changed the solution for half, discarding half of the old SPM2 medium, and adding half of the new SPM2 medium;

(4) Day 4, collected a portion of EB for flow detection, and the detection method is shown in Example 2;

Among them, A culture plate: normoxic (Day 1)+hypoxia (Day 0-Day 4), B culture plate: normoxic (Day 1)+normoxic (Day 0-Day 4), C culture plate: hypoxia (Day 1)+normoxic (Day 0-Day 4), D culture plate: hypoxia (Day 1)+hypoxia (Day 0-Day 4).

In this example, the inhibitor for TGF-β type I receptors ALK5, ALK4, and ALK7 is 1-6 μM SB431542, small molecule substances that can perform similar functions include but are not limited to: Galunisertib (LY2157299), LY2109761, SB525334, SB505124, GW788388.

The remaining differentiation steps are the same as Example 1. And on the 4th, 12th, and 40th day of differentiation, flow detection was used to detect the proportion of CD34+/CD235 cells under different culture conditions.

### 4. Experimental Results

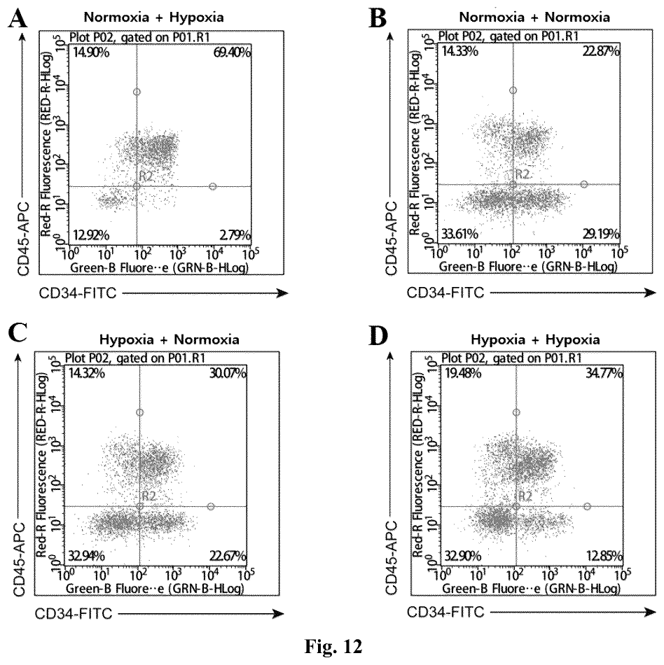

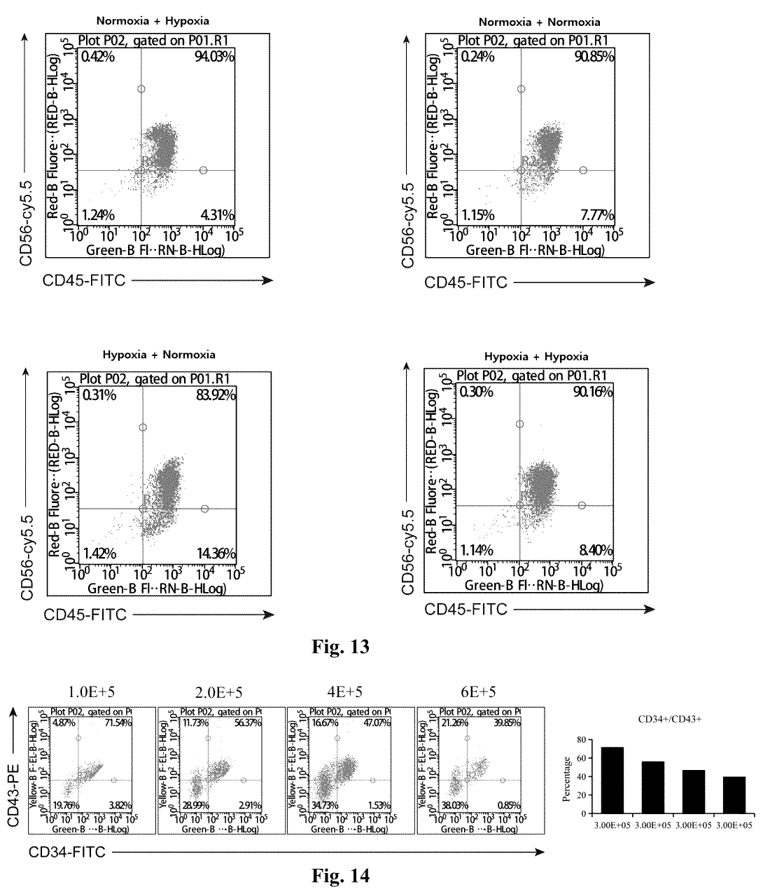

The detection results on the 4th day of differentiation under different experimental conditions are shown in FIGS. 11A-11D. The results show that on the 4th day of differentiation, the proportion of CD34+/CD235 cells is the highest under normoxic+hypoxia culture conditions; the detection results on the 12th day of differentiation under different experimental conditions are shown in FIGS. 12A-12D. The results show that on the 12th day of differentiation, the proportion of CD34+/CD45+cells in suspension cells under normoxic+hypoxia culture conditions is the highest; the results on the 40th day of differentiation under different experimental conditions are shown in FIGS. 13A-13D. The results show that on the 40th day of differentiation, the proportion of CD56+/CD45+cells in suspension cells is the highest under normoxic+hypoxia culture conditions. The above results indicate that the culture conditions of normoxia+hypoxia are significantly superior to those of normoxia+normoxia, hypoxia+hypoxia, and hypoxia+normoxia.

### Example 5 the Effect of Different Cell Densities on Inducing iPSC Differentiation to Obtain CD34+Cells

### 1. Experimental Method

In step (2) of “2. Formation of embryoid bodies” in Example 1, different cell densities are set: 1×10⁵, 2×10⁵, 4×10⁵, 6×10⁵/mL, the remaining steps are the same as Example 1.

On the 12th day of differentiation, flow cytometry detection was used to detect the expression of CD34 and CD43 under different initial cell densities.

### 2. Experimental Results

The results are shown in FIG. 14, which shows that as the cell density increases, the proportion of CD34 and CD43 double positive cells gradually decreases on the 14th day of differentiation. Among them, the differentiation efficiency of forming embryoid bodies with a cell density of 1×10⁵/mL is the highest, therefore, the preferred cell density range is 1×10⁵-2×10⁵/mL, most preferably 1×10⁵/mL.

### Example 6 the Effect of Different bFGF Concentrations on Inducing iPSC Differentiation to Obtain CD34+Cells

1. Experimental method

In the SPM1 medium of step (1) of the “4. Inducing Mesodermal Cells to Differentiate into CD34+Hematopoietic Endothelial Cells (HECs)” stage in Example 1, different bFGF concentrations were set: 0 ng/ml, 5 ng/mL, 10 ng/ml, 25 ng/ml, 50 ng/ml, 75 ng/ml, and 100 ng/ml. The remaining steps are the same as Example 1. On the 4th day of differentiation, cells were collected and the proportion of CD34+cells was detected by flow cytometry detection.

2. Experimental results

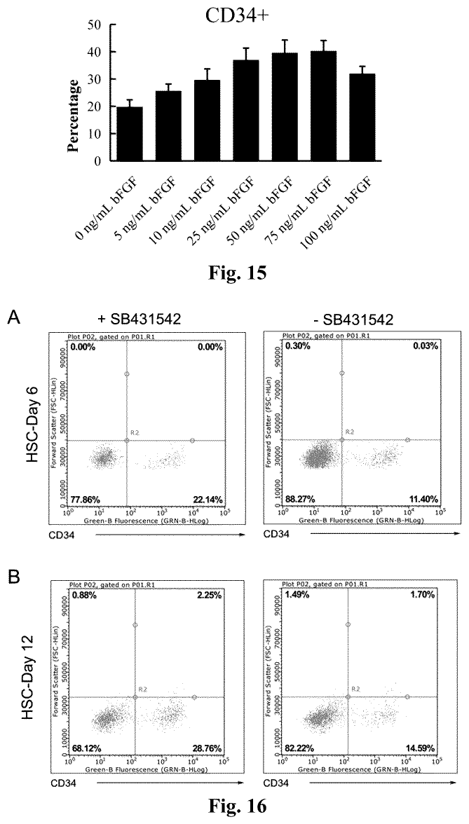

As shown in FIG. 15, the results show that when the concentration of bFGF is below 50 ng/mL, the proportion of CD34+cells increases with the increase of bFGF concentration. When the concentration of bFGF reaches 75 ng/ml and 100 ng/ml, the proportion of CD34+cells decreases with the increase of bFGF concentration. Therefore, the optimal concentration of bFGF is 25-75 ng/mL, and the most optimal is 50 ng/mL.

### Example 7 the Effects of Different Inhibitor Concentrations of TGF-β Type I Receptors ALK5, ALK4, and ALK7 on Inducing iPSC Differentiation into CD34+Cells

### 1. Experimental Method

In the SPM2 medium of step (2) of the “4. Inducing Mesodermal Cells to Differentiate into CD34+Hematopoietic Endothelial Cells (HECs)” stage in Example 1, different concentrations of SB431542 were set: 0 mM and 6 mM. The remaining steps are the same as Example 1. Cells were collected on the 6th and 12th day of differentiation, and the expression of CD34 was detected by flow cytometry.

### 2. Experimental Results

The results are shown in FIG. 16, which show that comparing the situation not adding inhibitor SB431542 of TGF-β type I receptor ALK5, ALK4, and ALK7, the addition of SB431542 on Day 2 significantly increased the proportion of CD34+cells on the 6th and 12th day of differentiation.

### Example 8 Comparison of the Expression of INK Obtained by the Present Invention and Umbilical Cord Blood Derived NK Biomarkers

### 1. Experimental Method

Separating NK cells derived from umbilical cord blood and iNK cells obtained on the 40th day of differentiation of the present invention, and detecting the expression of CD45, CD56, CD3, CD94, NKp30, NKp44, NKp46, TRAIL, Granzyme B by flow cytometry, respectively.

### 2. Experimental Results

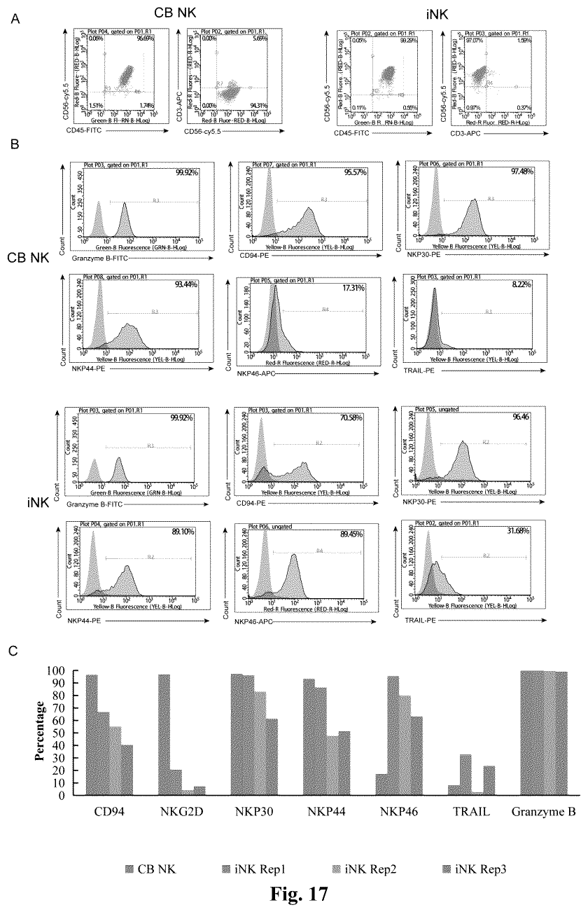

As shown in FIGS. 17A-17C, the results showed that the expression ratios of CD45 and CD56 in umbilical cord blood derived NK and differentiated iNK were 96% and 99%, respectively. There were about 5% of CD3+cells in umbilical cord blood derived NK, while about 1.5% of CD3+cells in iNK; both umbilical cord blood derived NK and iNK express Granzyme B, CD94, NKP30, NKP44, NKP46, TRAIL, and there is no significant difference in cell ratio. It can be seen that the marker detection results of iNK reach the level of umbilical cord blood NK, and the expression ratio of CD45 and CD56 is as high as 99%, which is higher than 96% of umbilical cord blood derived NK.

### Example 9 Comparison of Tumor Killing Ability Between INK Obtained by the Present Invention and Umbilical Cord Blood Derived NK (CB NK)

### 1. Experimental Method

Firstly, human prostate cancer LNCap-GFP cells (purchased from Procell company) were attached to the wall, with a 96 well plate containing 10,000 cells per well. Subsequently, NK cells derived from cord blood and iNK cells prepared by the present invention were taken separately, and effector NK cells were added at different target ratios of 0, 0.5, 1, 2, 4, and 8. Each target ratio was subjected to three repeated tests. The culture plate was placed in the incucyte monitoring instrument to detect GFP signals, and the killing curves of CB NK and iNK were obtained.

### 2. Experimental Results

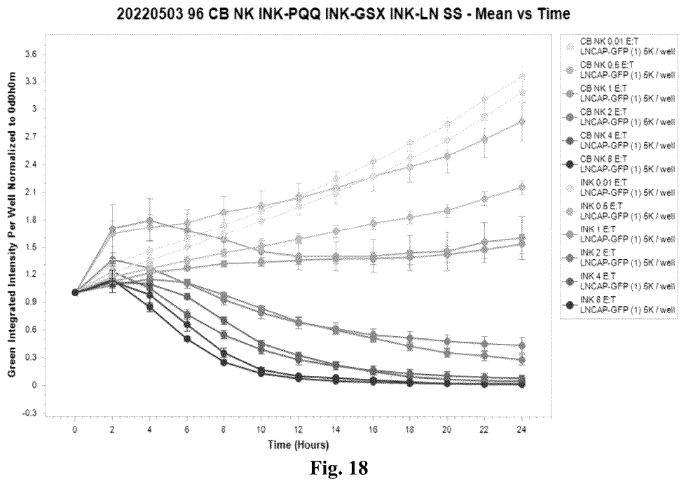

As shown in FIG. 18, the results show that CB NK and the INK prepared by the present invention have the same killing effect on target cells at an effective target ratio of 1:1 or greater. At an effective target ratio of 0.5, the iNK prepared by the present invention has stronger tumor killing ability than CB NK, indicating that the killing ability of the iNK prepared by the present invention on target cells is consistent or even stronger than that of NK derived from umbilical cord blood.

The explanation of the above embodiments is only for understanding the methods and core ideas of the present invention. It should be pointed out that for ordinary skilled person in this field, without departing from the principles of the present invention, improvements and modifications can be made to the present invention, which will also fall within the scope of protection of the claims of the present invention.

1. A culture medium for inducing iPSC differentiation to obtain CD34+cells, characterized in that the culture medium comprises a first stage medium, a second stage medium, a third stage medium, and a fourth stage medium;
the first stage culture medium is an E8 complete culture medium containing a ROCK pathway inhibitor and polyvinyl alcohol;
the second stage culture medium is an E8 complete culture medium containing a GSK-3B inhibitor;
the third stage culture medium comprises a SPM1 culture medium and a SPM2 culture medium;
the fourth stage culture medium is a SPM3 culture medium;
the SPM1 culture medium comprises stepro-34 complete culture medium, DMEM/F12 culture medium, L-glutamine, ascorbic acid, ITS-X, BMP4, VEGF, bFGF;
the SPM2 culture medium comprises the SPM1 culture medium and an inhibitor of TGF-β type I receptors ALK5, ALK4 and ALK7;
the SPM3 culture medium comprises stepro-34 complete culture medium, DMEM/F12 culture medium, L-glutamine, ascorbic acid, ITS-X, bFGF, VEGF, SCF, TPO, FLT-3L.

2. The culture medium according to claim 1, characterized in that the ROCK pathway inhibitor in the first stage culture medium is Y-27632;
the GSK-3β inhibitor in the second stage culture medium is CHIR-99021;
the inhibitor of TGF-β type I receptors ALK5, ALK4 and ALK7 in the third stage culture medium is SB431542;
a concentration of Y-27632 in the first stage culture medium is 0.5-20 μM;
a concentration of polyvinyl alcohol in the first stage culture medium is 2-6 mg/ml;
a concentration of CHIR-99021 in the second stage culture medium is 1-20 μM;
a concentrations of L-glutamine, ascorbic acid, ITS-X, BMP4, VEGF, bFGF and SB431542 in the third stage culture medium is (0.1-5) %, (10-100) μg/mL, (0.1-5)×, (10-100) ng/ml, (10-100) ng/ml, (10-100) ng/ml and (1-10) μM, respectively,
a concentration of L-glutamine, ascorbic acid, ITS-X, bFGF, VEGF, SCF, TPO and FLT-3L in the fourth stage culture medium is (0.1-5) %, (10-100) μg/mL, (0.1-5)×, (10-100) ng/mL, (10-100) ng/ml, (10-100) ng/ml, (10-100) ng/ml and (1-50) ng/ml respectively.

3. The culture medium according to claim 2, characterized in that a concentration of Y-27632 in the first stage culture medium is 10 μM;
a concentration of polyvinyl alcohol in the first stage culture medium is 4 mg/mL;
a concentration of CHIR-99021 in the second stage culture medium is 10 μM;
a concentrations of L-glutamine, ascorbic acid, ITS-X, BMP4, VEGF, bFGF and SB431542 in the third stage culture medium is 1%, 50 μg/mL, 1×, 50 ng/ml, 50 ng/ml, 50 ng/ml and 6 μM, respectively;
a concentrations of L-glutamine, ascorbic acid, ITS-X, bFGF, VEGF, SCF, TPO and FLT-3L in the fourth stage culture medium is 1%, 50 μg/mL, 1×, 50 ng/mL, 50 ng/mL, 50 ng/ml, 30 ng/ml and 10 ng/mL, respectively.

4. The culture medium according to claim 1, characterized in that the culture medium can also induce iPSC differentiation to obtain NK cells, the culture medium for obtaining NK cells comprises a first stage medium, a second stage medium, a third stage medium, a fourth stage medium and a fifth stage medium;
the first stage culture medium, the second stage culture medium, the third stage culture medium and the fourth stage culture medium are the first stage culture medium, the second stage culture medium, the third stage culture medium and the fourth stage culture medium according to claim 1;
the fifth stage culture medium is SPM-NK culture medium;
the SPM-NK culture medium comprises stepro-34 complete culture medium, DMEM/F12 culture medium, L-glutamine, ascorbic acid, ITS-X, SCF, Flt-3L, IL-3, IL-7 and IL-15;
a concentrations of L-glutamine, ascorbic acid, ITS-X, SCF, Flt-3L, IL-3, IL-7, and IL-15 in the SPM-NK culture medium are (0.1-5) %, (10-100) μg/mL, (0.1-5) ×, (10-50) ng/ml, (1-20) ng/mL, (1-10) ng/mL, (10-50) ng/mL, (1-100) ng/mL, respectively.

5. The culture medium according to claim 4, characterized in that a concentration of L-glutamine, ascorbic acid, ITS-X, SCF, Flt-3L, IL-3, IL-7 and IL-15 in the SPM-NK culture medium is 1%, 50 μg/mL, 1×, 20 ng/mL, 10 ng/mL, 5 ng/mL, 20 ng/mL, 50 ng/mL, respectively.

6. A method for inducing iPSC to differentiate into CD34+cells, characterized in that the method comprises the following steps:
(1) a first stage, Day 1, under normoxic conditions, suspension culturing iPSC using the first stage culture medium according to claim 1 to form embryoid bodies;
(2) a second stage, Day 0, under hypoxic conditions, inducing and culturing the embryoid bodies using the second stage culture medium according to claim 1 to form mesodermal cells;
(3) a third stage, Day 1-Day 4, under hypoxic conditions, inducing and culturing the mesodermal cells using the third stage culture medium according to claim 1 to form CD34+ hematopoietic endothelial cells;
(4) a fourth stage, Day 5-Day 12, under normoxic conditions, inducing and culturing the CD34+ hematopoietic endothelial cells using the fourth stage culture medium according to claim 1 to form CD34+/CD45+cells.

7. The method according to claim 6, characterized in that formation of the embryoid bodies in step (1) comprises the following steps: Day 1, digesting iPSC to a single cell state, inoculating cells, adding a first stage culture medium for resuspension culture, to form embryoid bodies;
a density of the inoculated cells is 1×10⁵-2×10⁵/mL;
formation of CD34+ hematopoietic endothelial cells in step (3) comprises the following steps:
(a) Day 1, inducing and culturing the mesodermal cells in SPM1 culture medium;
(b) Day 2, replacing the SPM1 culture medium with SPM2 culture medium for induction culture;
(c) Day 3, changing half of solution, discarding half of the old SPM2 culture medium, and adding half of the new SPM2 culture medium;
(d) Day 4, adherent culturing embryoid bodies to form CD34+ hematopoietic endothelial cells.

8. The method according to claim 7, characterized in that a density of inoculated cells in step (1) is 1×10⁵/mL;
cultivation conditions in step (1) are 5% CO₂ and 37° C. constant temperature cultivation;
cultivation conditions in step (2) are 5% CO₂, 90% N2, and 37° C. constant temperature cultivation;
cultivation conditions in step (3) are 5% CO₂, 90% N2, and 37° C. constant temperature cultivation;
cultivation conditions in step (4) are 5% CO₂ and 37° C. constant temperature cultivation.

9. The method according to claim 6, characterized in that the method can also induce iPSC to differentiate into NK cells, which further comprises the following: a fifth stage, Day 13-Day 40, under normoxic conditions, inducing and culturing the CD34+/CD45+cells using a SPM-NK culture medium to form NK cells.

10. The method according to claim 9, characterized in that the fifth stage comprises the following steps:
a) Day 13-Day 18, suspension culturing the CD34+/CD45+cells in the fifth stage culture medium;
b) Day 19-Day 40, replacing the fifth stage culture medium with a fifth stage culture medium without IL-3 for suspension culture to form NK cells;
the conditions for cultivation are 5% CO₂ and constant temperature cultivation at 37° C.

11. A cell population, characterized in that the cell population is a CD34+cell population or a derivative thereof, or an NK cell population or a derivative thereof, the CD34+cell population of the derivative thereof is induced by differentiation using the method according to claim 6;
the CD34+cell population simultaneously expresses CD45;
the derivative of the CD34+cell population is a hematopoietic cell line cell population obtained by inducing differentiation of the CD34+cell population;
the hematopoietic cell line cell population induced by inducing differentiation of the CD34+cell population includes T cells, NK cells, B cells and macrophages.

12. A pharmaceutical composition for treating and/or preventing hematological diseases and/or autoimmune diseases and/or solid tumors, the pharmaceutical composition comprises the CD34+cell population or its derivatives, NK cell population or its derivatives according to claim 11.

13. A method for
inducing iPSC differentiation to obtain CD34+cells, the method comprising utilizing the culture medium according to claim 1.

14. A method for inducing iPSC differentiation to obtain NK cells, the method comprising utilizing the culture medium according to claim 4.

15. A method for treating and/or preventing hematological diseases and/or autoimmune diseases and/or solid tumors, the method comprising utilizing the CD34+cell population or its derivatives and NK cell population or its derivatives according to claim 11.

16. The cell population according to claim 11, the NK cell population or the derivative thereof is prepared by inducing iPSC differentiation comprising a fifth stage, Day 13-Day 40, under normoxic conditions, inducing and culturing the CD34+/CD45+cells using a SPM-NK culture medium to form NK cells.
